(19) United States
(12) Patent Application Publication
(10) Pub. No.: US 2022/0047425 A1
(43) Pub. Date: Feb. 17, 2022

(54) CANDLING ASSEMBLY INCLUDING A DISK OPERATIVELY ATTACHED TO AN EAR CANDLE FOR USE WITH AN EAR

(71) Applicant: Richard J. Santos, Saginaw, MI (US)

(72) Inventor: Richard J. Santos, Saginaw, MI (US)

(21) Appl. No.: 17/403,819

(22) Filed: Aug. 16, 2021

Related U.S. Application Data
(60) Provisional application No. 63/066,169, filed on Aug. 15, 2020.

(51) Int. Cl. A61F 11/00; C11C 5/00

(52) U.S. Cl. CPC A61F 11/006; C11C 5/008

(57) ABSTRACT

A candling assembly for use within an ear canal of a person undergoing an ear candling process. The candling assembly includes an ear candle and a disk. The ear candle extends along a shaft between a first end and a second end. The disk is secured to the shaft, and the disk is movable between a closed position and an open position. The disk defines a pocket that opens toward the second end, when the disk is in the open position, configured to collect debris falling from the second end toward the ear canal of the ear and the user of the candling assembly. The disk is collapsed onto itself, with a portion of the shaft of the ear candle therebetween, when the disk is in the closed position. As such, the ear candling assembly is configured to be packaged for storage in a bag when not in use.

### CROSS REFERENCE TO RELATED APPLICATIONS

The subject patent application claims priority to and all the advantages of U.S. Provisional Patent Application No. 63/066,169, filed on Aug. 15, 2020, the contents of which are incorporated herein by reference in their entirety.

### TECHNICAL FIELD

The present disclosure is related to a candling assembly including a disk operatively attached to an ear candle, the cleaning assembly for use with an ear.

### BACKGROUND

Ear candling (sometimes referred to a “ear coning”) is a procedure used holistically or medically, as a complimentary therapy or treatment for a wide range of conditions. Ear candling uses ear candles, which are hollow tubes coated in wax that are inserted into a patients' ears and then lit at the far end. It is thought by some that the flame creates negative pressure, drawing wax and debris out of the ear.

Every person has cerumen, i.e., earwax. When the person has a sense of fullness, discharge from their ear, or has difficulty hearing they may need to remove a buildup of wax. Ear candling is one of the oldest practices used to get rid of excess ear wax.

Also, ear candling has been holistically used by some to open the spiritual centers and to clear and cleanse the physical, emotional, and other “auric” bodies of patients. Traditionally, candlings were performed by master energetic healers who worked on the physical as well as intermediate planes. The fire and smoke from the ear candles are used to cleanse and/or create the movement of energy for healing.

### SUMMARY

One possible aspect of the disclosure provides a candling assembly for use within an ear canal of an ear by a user undergoing an ear candling process. The candling assembly includes an ear candle and a disk. The ear candle extends along a shaft between a first end and a second end. The first end is configured for insertion into an ear canal of an ear and the second end is configured for being ignited, to subsequently burn along the shaft, toward the first end. The disk is secured to the shaft of the ear candle, and the disk is configured to move between a closed position and an open position. The disk defines a pocket that opens toward the second end, when the disk is in the open position. As such, the pocket is configured to collect debris falling from the second end toward the ear canal of the ear and the user of the candling assembly. The disk is collapsed onto itself, with a portion of the shaft of the ear candle therebetween, when the disk is in the closed position. As such, the ear candling assembly is configured to be packaged for storage in a bag when not in use.

The disk is formed with scored lines that are folded in a zig-zag or accordion pattern. This type of folding allows the disk to become collapsible, and thus the disk can be collapsed into a smaller form that may look like a “skirt.” A center of the disk is pierced with or without a loss of material. Without the loss of material, the disk becomes a flanged sleeve that allows the ear candle to move through the center opening or aperture. The flanges are used to attach or otherwise affix the disk to the ear candle.

The disk is folded or collapsed, positioned, and then attached to the ear candle that has been inserted and positioned through the center of the disk to form a candling assembly. The intent of the larger and collapsible ear candle disk that is pre-attached to the ear candle is to make the disk convenient, easier, quicker, and safer for the user to engage in the ear candling process. The candling assembly is removed from packaging, where the user unfolds or otherwise opens the pre-attached disk, relative to the ear candle to catch and/or hold ear candle wax drippings, ear candle ash remains, and/or any burned candle wax residue during the ear candling process. The pre-attached disk may be detached from the used ear candle once the ear candling process has ended, flattened, and then used as a flattened surface to receive ear candle contents, including, but not limited to, ear wax, other matter from the used ear candle, and the used ear candle itself onto the unfolded flattened disk prior to disposal into a trash receptacle.

Ear candle disks are small (about three inches in diameter) and thus may be unsafe for the user when ear candling. Disks used with ear candles have the potential of moving or sliding down the ear candle during the ear candling process, and thus may be undesirable for the user. Users have been known to use paper or plastic plates, pie pans, and other materials need to spend time looking for paper or plastic plates, pie pans, or other materials and creating their own homemade disks. Users who do not have the equipment at home to create their own homemade disks end up having to spend time traveling to a store to buy the materials, asking a neighbor or another person for materials, or forgoing the ear candling process all together. Further, some users may have physical limitations (e.g., arthritis, injury, and the like) that physically prevent them from being able to use any tools to create a homemade disk. Users who craft their own homemade disk may cut a hole too large that causes the disk to slide down the ear candle or rest on the person's ear, skin, hair, and/or other surrounding surface. Additionally, if too large of a hole is cut in the homemade disk, burned candle pieces or ash may fall between the large cut hole and ear candle and onto the person's ear and/or hair.

These types of homemade disks described above do not provide a disk that is of a large enough size, collapsible, and/or attached to the ear candle to assist with providing a surface to collect ear wax, burned ear candle pieces, unburned ear candle pieces, and/or other items for later disposal during the car candling process.

The present disclosure seeks to provide a ready-made disk for an ear candle that is of suitable size, collapsible, and attached to an ear candle for the user to use right out of the package, that helps to eliminate any fear factor or intimidation that would typically be associated with using an ear candle.

The above features and advantages and other features and advantages of the present teachings are readily apparent from the following detailed description of the best modes for carrying out the present teachings when taken in connection with the accompanying drawings.

### BRIEF DESCRIPTION OF THE DRAWINGS

Various features of the present disclosure will become more fully apparent from the following description and appended claims, taken in conjunction with the accompanying drawings. Understanding that these drawings depict only typical embodiments of the disclosure and are, therefore, not to be considered limiting of its scope, the embodiments of the disclosure will be described and explained with additional specificity and detail through the use of the accompanying drawings.

FIG. 1 is a schematic illustrative view of a candling assembly being applied to an ear canal of a person by a user to perform an ear candling process.

FIG. 2A is a schematic view of cardstock with dashed lines scored thereon to form a disk for the candling assembly.

FIG. 2B is a schematic view of the cardstock of FIG. 2A illustrating a circular shape for the disk being cut from the cardstock.

FIGS. 3-7 are schematic illustrative views of the circular shape of FIG. 2B, with a plurality of fold lines formed on, the disk in intersecting relationship to one another across a center.

FIGS. 8A-8B are schematic illustrative views of the disk, folded along the plurality of fold lines to form an accordion like structure.

FIGS. 9A-9E are schematic illustrative views of an aperture formed through the disk, where the plurality of fold lines intersect one another.

FIGS. 10A-10D are schematic illustrative views of the candling assembly of FIG. 1 in an open position.

FIGS. 11A-11D are schematic illustrative views of the candling assembly, with the disk secured to the ear candle via adhesive tape.

FIGS. 12A-12B are illustrative views of the candle assembly, with the disk in a closed position, and the candle assembly inserted within packaging.

FIG. 13 is a schematic perspective view of another embodiment of the candle assembly with the disk attached to the ear candle via a sleeve.

FIGS. 14A-14B are schematic illustrative views of the disk of the candle assembly detached from the ear candle after use, with the disk in a flattened position and the aperture closed via the tape to illustrate the disk capturing debris from the ear candle, prior to disposal within the disk.

### DETAILED DESCRIPTION

The components of the disclosed embodiments, as described and illustrated herein, may be arranged and designed in a variety of different configurations. Thus, the following detailed description is not intended to limit the scope of the disclosure, as claimed, but is merely representative of possible embodiments thereof. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure. Furthermore, the drawings are in simplified form and are not to precise scale. For purposes of convenience and clarity only, directional terms such as top, bottom, left, right, up, down, upper, lower, upward and downward may be used with respect to the drawings. These and similar directional terms are not to be construed to limit the scope of the disclosure in any manner. Additionally, the disclosure, as illustrated and described herein, may be practiced in the absence of any element that is not specifically disclosed herein.

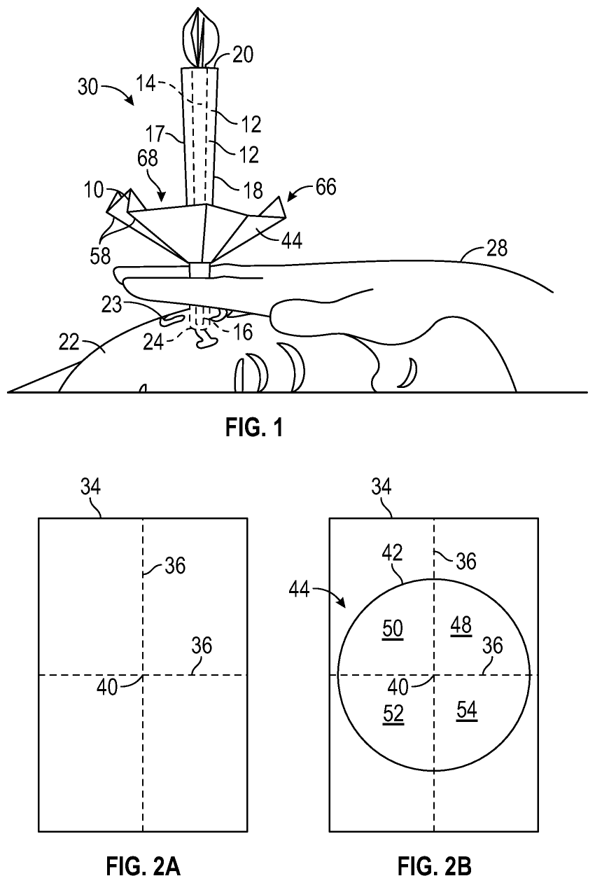

Referring to the drawings, wherein like reference numbers refer to like components throughout the several Figures, a disk 10 for use with an ear candle 12 is shown in FIG. 1. The ear candle 12 defines a hollow interior 14, resembling a hollow straw, with a first end 16 that is generally tapered. The ear candle 12 is coated in wax 18, and extends along a shaft 17, leaving the first end 16 (bottom end or tapered end) and a second end 20 (top end) open. In some non-limiting examples, the ear candles 12 may be approximately 10 inches in length, and 0.75 inches in diameter. It should be appreciated that ear candles having different lengths and diameters may be used, as known to those of skill in the art. The ear candle 12 may be formed from strips of cloth, e.g., muslin cloth and the like, that are dipped in wax, and coiled to form a long slender cone, defining the hollow interior 14.

During the ear candling process, a person 22 lies on his or her side with the tapered end 16 resting gently, but snugly in an ear canal 24 of the person 22. Cloth at a top end 20 of the ear candle 12 is lit by a second person 28 (user). As the ear candle 12 slowly burns down, and since hot air rises, heat created by the burning ear candle creates a slight, warm swirling current of air, and a slight, relative vacuum. Some people indicate that the relative vacuum causes the person's ear wax to be drawn into the hollow of the ear candle. For others, it is believed that many benefits to the person undergoing ear candling are the result of heat and energetic stimulation of person's reflexes.

Referring to FIG. 13, a candling assembly 30 includes the disk 10 attached to the ear candle 12, such that the candling assembly 30 is removed from packaging 32 with the disk 10 already attached to the ear candle 12. The ready-made candle assembly 30, i.e., the disk 10 already attached to the ear candle 12, helps to eliminate any fear factor or intimidation that would typically be associated with using an ear candle. As such, when the candling assembly 30 is removed from the packaging 32, the user 28 is able to open up the disk 10, relative to the ear candle 12, without having to assemble or otherwise attach the disk 10 to the ear candle 12.

FIGS. 2A-11D illustrate a non-limiting example of the formation of the disk 10, prior to attachment to the ear candle 12. Referring to FIGS. 2A and 2B, the disk 10 may be formed from a rectangular sheet of cardstock 34 and/or other material.

The ear candle 12 disk 10 may be made from yellow 65 lb. cardstock 34. However, it should be appreciated that the disk 10 can be made from other materials (e.g., cardboard, soft plastic, paper, etc.). Further, the disk 10 may be formed from a material that is fire resistant or be coated in a fire-retardant material. The cardstock 34 and other material used to form the disk 10 may vary in weight (e.g., 60 lb., 70 lb., lower or higher weight lb., etc.). The disk 10 may be formed from cardstock 34 having any desired color or color combination.

The cardstock 34 is scored in half vertically and horizontally as denoted by the dashed lines 36 in FIG. 2A. An aperture 38 (hole) is formed through the cardstock 34 where the dashed lines 36 intersect, as denoted by the dot 40 in FIG. 2A. The dashed lines 36 and aperture 38 may be formed by a device, machine, or object, as known to those of skill in the art.

With reference to FIG. 2B, a circle cutter device may be positioned where the dot 40 is illustrated. The circle cutter device is used to cut a circular cutout 44 through the cardstock 34, as denoted by the circular shape 42 in FIG. 2B. By way of a non-limiting example, a diameter of the circular shape 42 may be about 8.25 inches. The disk 10 shape diameter may vary, as required. The pierced aperture 38 may define a diameter of 0.5 inches, to allow a 0.5-inch diameter ear candle 12 to freely move through the aperture 38 of the disk 10. The diameter of the aperture 38 may vary, to allow candles 12 of different diameters. However, it should be appreciated that the diameter of the circular shape 42 may be smaller or larger, as desired. It should also be appreciated that the circular shape 42 may be formed by any device or machine, as known to those of skill in the art.

The profile or shape of the disk 10 shape illustrated in the foregoing examples is circular 44. It should be appreciated that the profile of the disk 10 may vary (e.g., octagon, decagon, etc.) and/or an edge of the disk 10 shape edge may also vary (e.g., scalloped, wavy, zig zag, etc.), without departing from the scope of the disclosure.

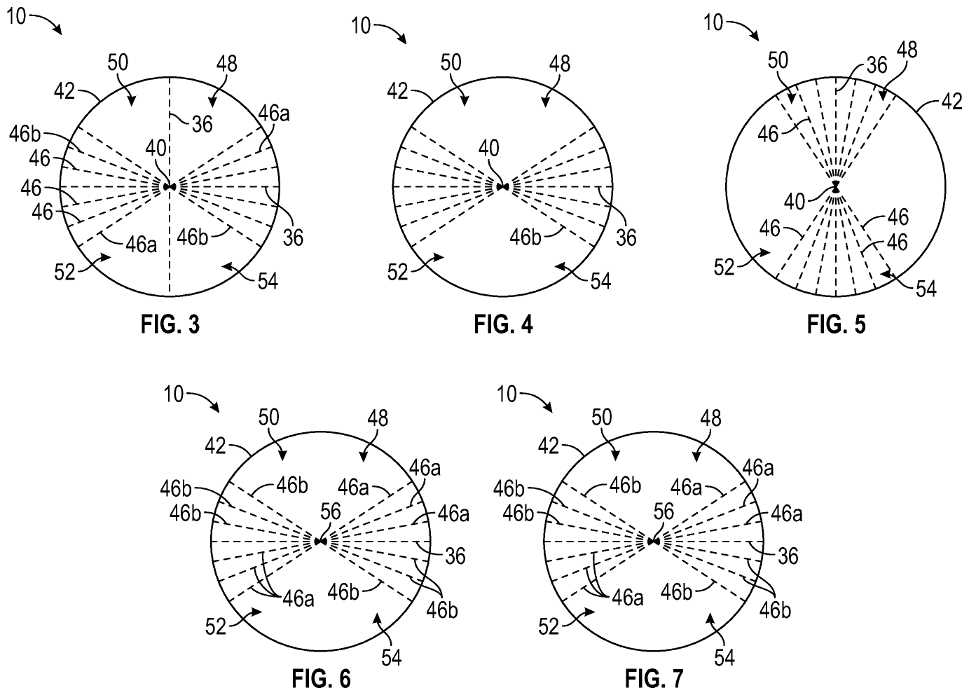

Referring now to FIGS. 3-7, the dashed lines 36 of the circular shape 42 are illustrated as intersecting to divide the circular cutout 44 into four quadrants, i.e., a first quadrant 48, a second quadrant 50, a third quadrant 52, and a fourth quadrant 54. A plurality of fold lines 46 are shown as being formed in the circular cutout 44. The fold lines 46 are scored diagonal lines that extend across the diameter and intersect one another across the aperture 38. As such, three separate fold lines 46, i.e., first fold lines 46a, extend from the first quadrant 48 to the third quadrant 52, and three other fold lines 46, i.e., second fold lines 46b, extend from the second quadrant 50 to the fourth quadrant 54. The first fold lines 46a and the second fold lines 46b intersect one another across the aperture 38. It should be appreciated that the first and the second fold lines 46a, 46b may be made prior to, or after, cutting the circular shape 42. It should also be appreciated that the number of fold lines 46 may vary in number, and may or may not be equally spaced from each other, relative to the dashed lines 36. The fold lines 46 may be made by a device or machine, as known to those of skill in the art.

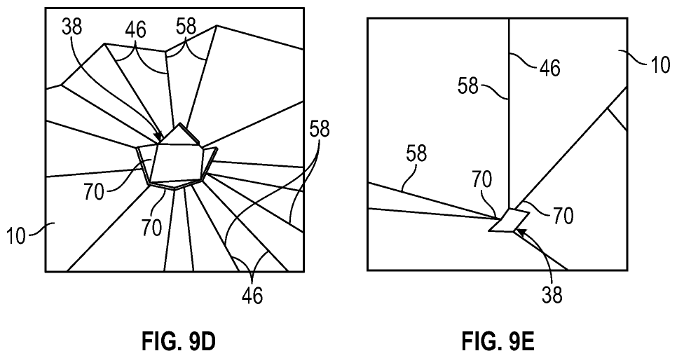

With reference to FIG. 6, the aperture 38 may be pierced through the circular cutout 44 as a plus (+) sign 56. In one non-limiting example, the aperture 38 provides a center opening, i.e., aperture 38, configured to allow the ear candle 12 to move or pass therethrough, as shown in FIGS. 9A-11D. The cut plus sign 56 that is made may vary in design (e.g., an asterisk (*), double plus sign (++), etc.) and the aperture 38 size or diameter may similarly vary in size, e.g., may be smaller or larger than 0.5 inches. The aperture 38, i.e., the cut plus sign 56 or other design variation cuts, may be made prior to or after cutting the circle or disk shape by a device or machine, as known to those skilled in the art. Therefore, a device or machine may be used to cut the circular shape 42, score the dashed line(s) 36 and the fold lines 46, where indicated in FIGS. 3-7, and to cut the plus sign 56 to form the aperture 38 through the material, as illustrated in FIGS. 6-7. The cut circle 44, scored lines 36, and cut plus sign 56 may or may not be done in one process. As illustrated in FIGS. 6-7, in one non-limiting example, the vertical dashed line 36 (as shown in FIGS. 2A, 2B, 3) may not be made at all.

With reference to FIG. 5, the process of cutting the circular shape 42 and scoring the dashed lines 36 may be done in a horizontal orientation as shown in FIG. 4 or done in a vertical orientation as shown in FIG. 5, and they may also be done in various diagonal orientations. The cut circle 44 and scored dashed lines 36 may or may not be done in one process. A scored horizontal dashed line as shown in FIGS. 2A, 2B, 3, 4 may not be made in FIG. 5.

Referring to FIG. 6, a center of the cut circle 44 is pierced to form the aperture 38 the material with a plus sign (+) cut, as denoted by the red plus sign in FIG. 6. In one non-limiting example, a size of the aperture 38 is about 0.5 inches in diameter to allow an ear candle to move or pass through its center as illustrated in FIGS. 9A-11D. It should be appreciated that since the aperture 38 is sized to receive the ear candle 12 therethrough, the size of the aperture 38 is dependent on a corresponding diameter of the ear candle 12. It should also be appreciated that the plus sign cut 56 may be shaped in other ways, e.g., an asterisk (*), etc. The plus sign cut 56 and/or other design variation cuts may be made prior to cutting the circular shape 42 by a device or machine. The cut circle 42, the scored dashed lines 36, and the cut plus sign 56 may or may not be done in one process.

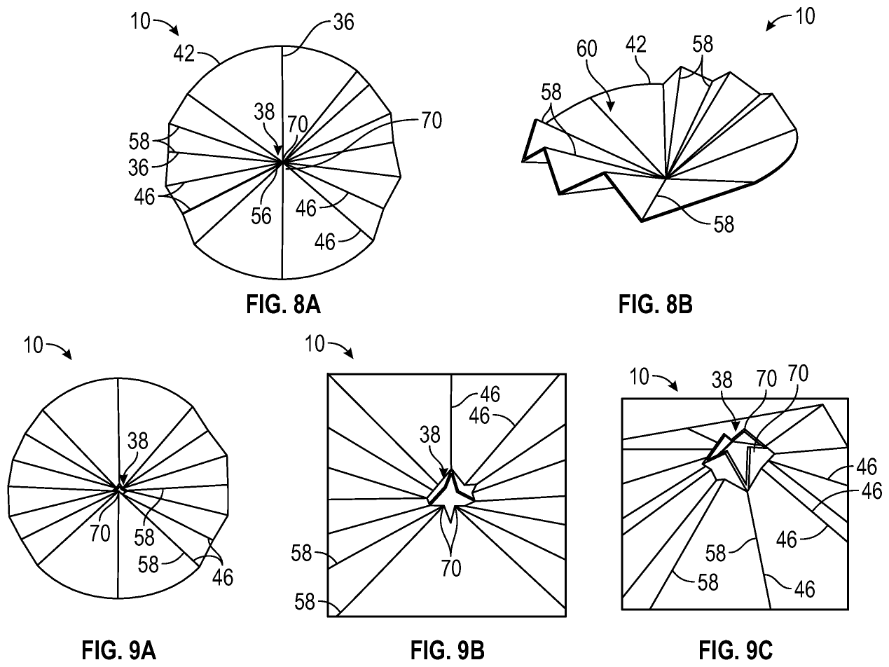

FIG. 7 illustrates the fold lines 46, along with a scored horizontal dashed line 36 that are formed in order to fold or crease back and forth to create a zig-zag or accordion-like pattern that allows the circular disk 42 to be folded or collapsed into a smaller shape or form, as illustrated in the non-limiting examples in FIGS. 8A-12B. The scored lines 36 and fold lines 46 or creases may vary in amount or number. The space or distance (i.e., angle) between each scored line may or may not be configured such that the scored lines 36 and/or fold lines 46 are equally spaced from one another other.

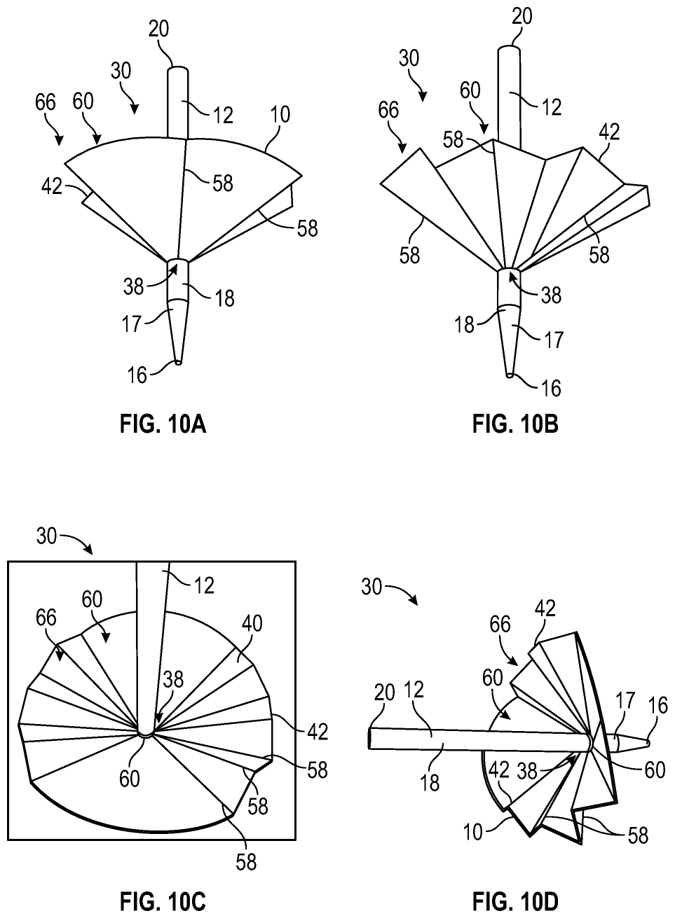

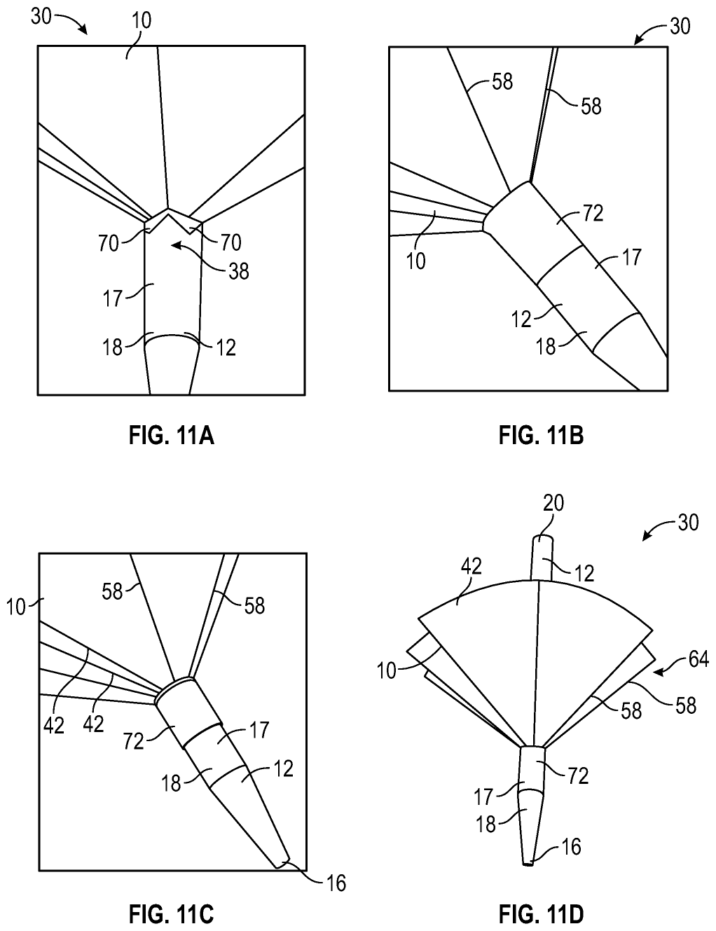

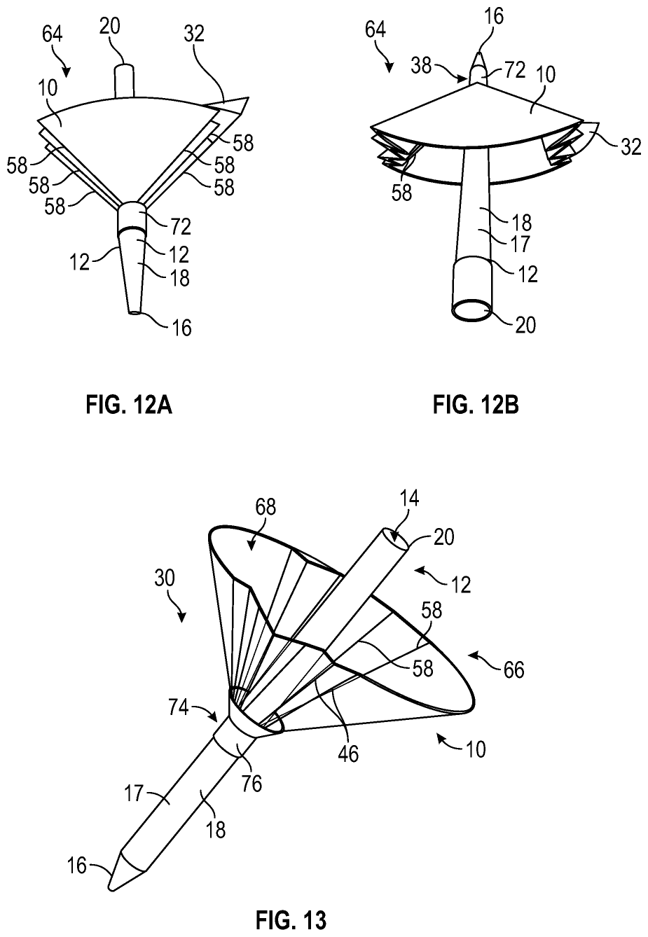

With reference to FIGS. 8A-8B, the disk 10 is shown to illustrate zig-zag or accordion-like folds 58 or creases resulting from folding the circular cutout 44. The folds 58 may or may not be done in the same process that forms the cut circle 42, the scored lines 36, the other fold lines 46, or the cut plus sign 56. The folds 58 made from one end of the circular cutout 44 through the center and to the opposite end of the circular cutout 44 create a zig-zag or accordion-like pattern that allows the circle or disk shape to be folded onto itself until collapse (FIGS. 11D, 12A, 12B), in order to form a profile that is compact and flattened, as compared with the circular cutout 44 (FIGS. 3-7) or the uncollapsed disk 10 with the folds 58 (FIGS. 1, 10C, 10D), that is of a smaller shape or form.

Referring now to FIGS. 9A-9E, once the plus sign cut 56 is formed in a center of the circular cutout 44, the center of the circular cutout 44 opened to form the aperture 38. The plus sign cut 56 in the center of the circular cutout 44 is opened to create the aperture 38 in the center thereof. The plus sign cut 56 may be opened by a device or machine to create the aperture 38 that allows the ear candle 12 to be inserted therein (FIGS. 10A-12B). Formation of the aperture 38 may be done before or after the formation of the folds 58 to create the zig-zag pattern described above.

FIGS. 10A-10D illustrates the aperture 38 allowing the ear candle 12 to be inserted through the center of the disk 10. In one non-limiting example, a device or machine is used to insert the ear candle 12 through the aperture 38. In another non-limiting example, the ear candle 12 is manually inserted through the aperture 38 by a person. FIG. 10 also shows the circle or disk shape in an open position with an inserted ear candle. Referring specifically to FIGS. 10C, 10D, an indicator 60 is operatively attached to the ear candle 12, between the disk 10 and the top end 20. The indicator 60 is configured to alert the user 28 when the ear candle 12 has burned to the point that the indicator 60 is at the top end 20 of the ear candle 12, and there is no unburned ear candle 12 between the indicator 60 and the top end 20 thereof. As such, the indicator 60 functions as a “Stop Here” burn line indicator. In one non-limiting example, the indicator is red in color. Additionally, the indicator 60 may provide different wordings or phrases, and may be placed in various distances from the second end 20 of the ear candle 12. The indicator 60 may be attached to the ear candle 12 and/or disk 10 by a device or machine. In another non-limiting example, the indicator 60 is attached to the ear candle 12 by hand. Other materials and/or ways may be applied or used to incorporate the indicator onto the ear candle 12 and/or circular cutout 44.

With reference to the Figures, the candling assembly 30 is movable between a collapsed, closed, or non-use position 64 (FIGS. 11D and 12A-12B) and an open or in-use position 66 (FIGS. 1 and 10A-10D). When the candling assembly 30 is in the in-use position 66, the disk 10 is open or otherwise unfurled relative to the ear candle 12. As such, the disk 10 defines a pocket 68 that opens toward the second end 20 of the ear candle 12. During use of the candling assembly 30, as illustrated in FIG. 1, the user 28 grasps the ear candle 12 between the disk 10 and the first end 16 thereof. The pocket 68 of the disk 10 functions to collect debris or ashes that may fall toward the person 22 receiving the ear candling or the user 28 performing the ear candling, while the first end 16 is in the ear canal 24 and the ear candle 12 is burning, in order to protect the user 28 and the person 22 receiving the ear candling, and to prevent unwanted messes. Therefore, the disk 10 attached to the ear candle 12 can be unfolded to provide a wide surface area to catch and/or hold ear candle 12 wax drippings, ear candle 12 ash remains, and/or any burned candle wax 18 residues during the ear candling process.

Referring now to FIGS. 11A-11D, the disk 10 is illustrated such that material 70 of the circular cutout 44, resulting from the formation of the cut plus sign 56, is moved in a direction that is away from, or opposite, the pocket 68. With reference to FIGS. 9A-9E and 11A, the material 70 may be four triangular shaped flanges that flare out outward, and define the aperture 38. Displacement of the material 70 in this direction allows the ear candle 12 to be inserted through the aperture 38, while allowing the material 70 to be positioned against the ear candle, between the first end 16 and the pocket 68 defined by a remaining portion of the disk 10. The cut plus sign material 70 of the disk 10 may provide a platform that allows the disk 10 to be taped, using an adhesive tape 72, or otherwise secured to the ear candle 12. The tape 72 may be used to secure the disk 10 to the ear candle 12 at any desired distance from the first end 16 (tapered end). Various adhesive applications may be used to attach or secure the circle or disk shape opening or aperture material to an inserted ear candle. In another non-limiting example, a band (i.e., an elastic band, mechanical band, and the like) may be used to secure the disk 10 to the ear candle 12, via the material 70. In yet another non-limiting example, an adhesive material is applied between the material 70 of the disk 10 and the ear candle 12 to secure the disk 10 to the ear candle 12. The disk 10 may be taped to the ear candle 12 that is inserted through the disk 10 center opening with various tapes (i.e., electrical, gaffer, etc.) where the disk 10 center opening material comes into contact with the ear candle 12. The disk 10 may also be secured to the ear candle 12 that is inserted through the aperture 38 of the disk 10 with an adhesive (i.e., glue, etc.) where the disk 10 center opening material comes into contact with the ear candle 12. The disk 10 may utilize a combination of tape 74 and adhesive to attach or secure the disk 10 to the ear candle 12 that is inserted through the disk 10 center opening. It should be appreciated that a device or machine may be used to apply the adhesive tape(s) 72 and/or other adhesive or mechanical applications to attach or secure the disk 10 to the ear candle 12 via the material 70.

FIGS. 12A-12B illustrate the candling assembly 30 in the non-use position 64, and disposed within packaging 32. As illustrated, the disk 10 attached or secured to the ear candle 12 is folded onto itself and collapsed into a smaller, more compact shape, to allow for shipment, storage, and/or merchandising display. The more compact shape of the non-use position 64 provides a disk 10 that resembles a “skirt” to allow the disk 10 to not only be attached to the ear candle 12, but also allows the resulting candling assembly 30 to fit conveniently into the packaging 32. It should be appreciated that a device or machine may be used to package the candling assembly 30, when in the non-use position 64. In another non-limiting example, the candling assembly 30 may be inserted in the packaging by hand. Additionally, various packaging styles may be used to package the folded or collapsed disk 10, i.e., when in the non-use position 64, that is attached or secured to the inserted ear candle 12.

Referring again to FIG. 13, the aperture 38 of the disk 10 may include a sleeve 74 with a flanged end 76 or lip that can be partly inserted through the center-opening or aperture until the flanged end or lip meets the material 70. Also, the aperture 38 of the disk 10 may be placed on top of an opening of the flanged end 76 of the sleeve 74, such that the aperture 38 of the disk 10 is aligned with an opening of the flanged sleeve 74. The flanged sleeve 74 may be made from various materials, or a combination of materials, and may be attached or otherwise secured to the disk 10, at or proximate the aperture 38, by various adhesive applications. The flanged sleeve 74 may be made partly by a material, such that the material itself is able to hold or secure an inserted ear candle in its place. A material such as rubber or an elastic stretchable material may be used to hold or secure an inserted ear candle in its place without using any adhesive applications. Various combinations to attach or secure the flanged sleeve to the circle or disk shape center-opening or aperture may be applied. For example, the flanged lip may be attached to the circle or disk shape center-opening or aperture by an adhesive application and the sleeve part of the flanged sleeve may use a rubber or elastic/stretchable material whereby an adhesive application is not needed. With the flanged sleeve attached or secured to the circle or disk shape, the flanged sleeve is able to allow an ear candle to be inserted through its center-opening as shown in FIG. 13. The disk 10, with the attached or secured flanged sleeve 74, is configured to move up and down an ear candle, between the ends 16, 20, and may be attached or otherwise secured to the ear candle 12 at various distances from the ear candle tip 16. A device or machine may be used to attach or secure the flanged sleeve 74 to the aperture 38. In other non-limiting examples, the flanged sleeve 74 is secured or otherwise attached to the aperture 38 by hand. Likewise, a device or machine may be used to apply adhesive tape(s) or other adhesive applications to attach or secure the flanged end 76 of the flanged sleeve 74 to the ear candle 12 inserted therein. A device or machine may be used to insert an ear candle through the center of the flanged sleeve 74 that does not need any adhesive applications to attach or secure the flanged sleeve to an inserted ear candle.

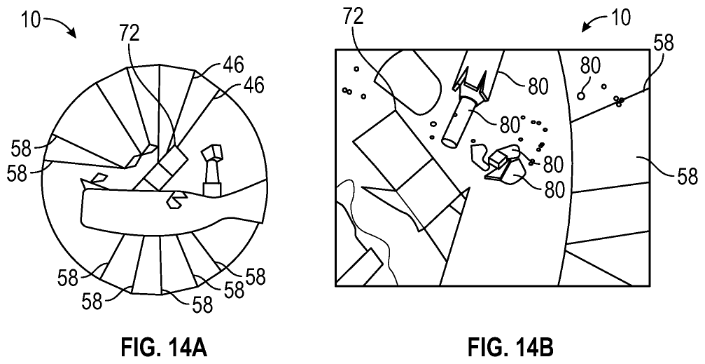

FIGS. 14A and 14B illustrates that the disk 10 attached to the ear candle 12 to form the candling assembly 30, may be separated or detached from the ear candle 12 by removing the adhesive tape(s) 72 and sliding the ear candle 12 through and out of the aperture 38 of the disk 10. The cut plus sign material 70 may be folded toward one another (i.e., toward the center) to close the aperture 38. As illustrated, the adhesive tape(s) 72 that was removed from candling assembly 30 may be subsequently applied over the closed aperture material 70 to keep the aperture 38 from opening. With the center opening or aperture closed, the circle or disk shape may be flattened and used as a surface to collect the contents of the ear candle such as ear wax, safety tip, ashes if applicable, the candle itself, and other items in one place prior to discarding the flattened circle or disk shape with the collected items 80, as shown in the photo examples in FIG. 14.

While the best modes for carrying out the many aspects of the present teachings have been described in detail, those familiar with the art to which these teachings relate will recognize various alternative aspects for practicing the present teachings that are within the scope of the appended claims.

1. A candling assembly for use within an ear canal of an ear by a user, the candling assembly comprising:
an ear candle extending along a shaft between a first end and a second end, the first end configured for insertion into an ear canal of an ear and the second end configured for being ignited, such that the ear candle burns along the shaft, toward the first end; and
a disk secured to the shaft of the ear candle, the disk configured to move between a closed position and an open position;
wherein the disk defines a pocket that opens toward the second end when the disk is in the open position, such that the pocket is configured to collect debris falling from the second end toward the ear canal of the ear and the user of the candling assembly; and
wherein the disk is collapsed onto itself, with a portion of the shaft of the ear candle therebetween, when the disk is in the closed position, such that the ear candling assembly is configured to be packaged for storage in a bag.

2. The candling assembly of claim 1, further wherein the disk is attached to the shaft of the ear candle via adhesive tape.

3. The candling assembly of claim 1, wherein the disk defines an aperture, and the ear candle extends through a center of the disk;
wherein the disk defines a plurality of fold lines that intersect one another at the center of the disk; and
wherein the disk is folded along the fold lines in alternating folds to form an accordion pattern.

4. The candling assembly of claim 3, wherein the disk is circular shaped when in the open position.

5. The candling assembly of claim 3, wherein the aperture is a slot within the disk, such that a portion of a material of the disk, proximate the slot, folds relative to another portion of a material of a remainder of the disk, to at least partially surround the aperture; and
wherein the material of the disk, proximate the aperture, is in contact relationship with the shaft.

6. The candling assembly of claim 5, wherein the material of the disk, proximate the aperture, is secured to the shaft via adhesive tape.

7. The candling assembly of claim 5, wherein the ear candle includes an indicator disposed on the shaft, between the aperture of the disk and the second end of the ear candle, wherein the indicator is configured to provide indication to a user that the ear candle is at a burn limit.

8. The candling assembly of claim 5, wherein the disk is formed from cardstock material.

9. The candling assembly of claim 8, wherein the cardstock material is at least a 65-pound cardstock material.

10. The candling assembly of claim 8, wherein the fold lines are folded along scored lines, formed in the cardstock material prior to folding along the fold lines.

11. The candling assembly of claim 6, wherein the disk is detachable from the shaft of the ear candle to provide a surface for collecting debris and materials from the ear candling process prior to disposal.

12. The candling assembly of claim 11, wherein the disk is detachable from the shaft of the ear candle as a result of removing the tape, such that the removed tape can be subsequently used to cover the aperture defined in the disk.
